(19) United States
(12) Patent Application Publication
(10) Pub. No.: US 2006/0198856 A1
(43) Pub. Date: Sep. 7, 2006

(54) Ibuprofen suspension stabilized with docusate sodium

(76) Inventor: Keith Whitehead, Oldsmar, FL (US)

Correspondence Address: WYATT BARTON PRATT, 15 COLUMBIA AVENUE, HOPEWELL, NJ, 08525, US

(21) Appl. No.: 11/069,713

(22) Filed: Mar. 1, 2005

(51) Int. Cl. A61K 31/192; A61K 9/00

(52) U.S. Cl. 424/400; 514/570; 514/569

(57) ABSTRACT

A stabilized aqueous pharmaceutical ibuprofen composition of ibuprofen stabilized with a stabilizing effective amount of docusate sodium.

### BACKGROUND OF THE INVENTION

1. Field of The Invention

The present invention pertains to ibuprofen suspensions stabilized with docusate sodium.

2. Brief Description of the Related Art

Ibuprofen, chemically known as (±)-2-(p-isobutylphenyl)propionic acid, is a well known medication that is difficult to formulate in aqueous suspensions. Docusate sodium is chemically known as bis(2-ethylhexyl) sodium sulfosuccinate, and also as dioctyl sodium sulfosuccinate and sulfo-butanedioic acid 1,4-bis(2-ethylhexyl)ester.

There is a need in the art to improve the stability of ibuprofen compositions. The present invention addresses this and other needs.

### SUMMARY OF THE INVENTION

The present invention includes a stabilized pharmaceutical suspension comprising a suspension having a pharmaceutically effective amount of ibuprofen and an amount of docusate sodium effective to stabilize the ibuprofen in suspension. The stabilized pharmaceutical suspensions of the present invention may be administered in unit dosage forms, and are particularly useful in pediatric formulations.

The present invention also includes a process for producing a pharmaceutically acceptable suspension of ibuprofen comprising the steps of preparing an aqueous ibuprofen composition and blending the aqueous ibuprofen composition with an effectively stabilizing amount of docusate sodium to produce a stabilized ibuprofen suspension thereof.

Surprising it has been discovered that the stability of ibuprofen formulations, particularly suspensions thereof for oral administration, may be substantially enhanced by the addition of a stabilizing effective amount of docusate sodium in the formulation.

Other features, advantages and embodiments of the invention will become apparent to those of ordinary skill in the art by the following description, accompanying examples and appended claims.

### DETAILED DESCRIPTION OF THE INVENTION

The aqueous pharmaceutical suspension of the present invention includes a pharmaceutically effective amount of ibuprofen together with an effective amount of docusate sodium to stabilize the ibuprofen. The stabilized ibuprofen compositions of the present invention provide suspensions having suitable shelf-life for pharmaceutical use, such as that defined by the United States Food and Drug Administration or other such criteria. Representative times include the ibuprofen compositions remaining stable for about eighteen months, two years, thirty months, three years, and the like, under actual or test storage conditions, including ambient and/or accelerated conditions, such as criteria under temperatures ranging from about 2° C. to about 40° C., increased relative humidity conditions, etc., for example storage conditions of 40° C. and 75% relative humidity for 3 months representing commercial storage of 24 months at 25° C., etc.

The aqueous formulations of the present invention, also referred to herein as suspensions, provide formulations for oral administration that is conveniently prepared by adding an aqueous solution of ibuprofen together with docusate sodium, with other excipients preferably added. The aqueous formulations herein provide a novel suspension system particularly well suited for use in stable and pourable ibuprofen pharmaceutical formulations, particularly in pediatric formulations. Preferred embodiments of the present invention include the elimination of certain known stabilizing compounds, such as ethanol, polyoxyethylene sorbitan monoleate, pregelatinized starch, and the like. For example, compounds known to be detrimental in pediatric formulations, such as ethanol, may be eliminated from pediatric formulations of the present invention while maintaining a stabilized ibuprofen suspension, with such exclusion of detrimental compounds determinable by one skilled in the art in light of the disclosure herein.

The suspensions of the present invention include an appropriate amount of ibuprofen generally having an amount that is therapeutically effective in a convenient dosage unit for a given patient. Preferred amounts of ibuprofen contained within the pharmaceutical formulation of the present invention include, for example without limitation, up to about 50 grams of ibuprofen per 1000 mL of suspension, with preferred ranges of from about 0.25 grams to about 40 grams of ibuprofen per 1000 mL of suspension, more preferably from about 0.5 grams to about 25 grams of ibuprofen per 1000 mL of suspension, still more preferably from about 0.75 grams to about 10 grams of ibuprofen per 1000 mL of suspension, and most preferably from about 1 gram to about 5 grams of ibuprofen per 1000 mL of suspension, such as 1.5 grams, 2 grams, 3 grams, 4 grams, etc. As the relative amount of ibuprofen is increased within a given volume of suspension, such as over about 20 grams of ibuprofen per 1000 mL, the suspension becomes increasing problematic to readily taste-mask with the addition of sweeteners and flavoring agents. Preferably the present invention includes a unit dosage of ibuprofen in an amount of from about 0.5% wt/vol. to about 5% wt/vol, more preferably from about 1% wt/vol to about 2% wt/vol, still more preferably from about 1.33% wt/vol to about 1.6% wt/vol.and most preferably about 1.33% wt/vol. The ibuprofen within the suspensions includes uniformly distributed, finely dispersed ibuprofen particles, and as such the present invention generally avoids agglomerations or other massings of the ibuprofen particles within the suspension. With such distribution of the ibuprofen together with the docusate sodium, it has been found that the resilience, non-degradation and other measures of stability of the ibuprofen are particularly appropriate for use in pharmaceutical formulations.

The suspensions of the present invention may include other component suitable for inclusion in the ibuprofen/docusate sodium suspension, such as active agents or excipients. Generally, particulate solids include particles sizes suitable for use in suspension, such as having a preferred particle diameter in the range of from about 1 micron to about 850 microns, with more preferred particle diameters ranging from about 37 microns to about 420 microns (400 to 40 mesh based on U.S. standard mesh screens), with the appropriate particle size being determinable by one skilled in the art, particularly as varied with the density of the particulate solid as a factor of Stokes' Law. When used herein, the terms “active agent”, “active pharmaceutical ingredient”, “pharmaceutical actives”, “API”, “active compound”, “therapeutic agent”, “therapeutic ingredient”, “therapeutic compound” and other like terms are used interchangeably and include salts and other pharmaceutical forms of the detailed compounds. Excipients may include pharmaceutically acceptable additives for proper formulation of a pharmaceutical suspension such as those detailed below.

Docusate sodium, chemically known as bis(2-ethylhexyl) sodium sulfosuccinate, and also as dioctyl sodium sulfosuccinate and sulfo-butanedioic acid 1,4-bis(2-ethylhexyl)ester, sodium salt is incorporated into the suspension. Docusate sodium is a hygroscopic anionic surfactant, that is a white or almost white, wax-like, bitter tasting, plastic solid having a octanol-like odor. Docusate sodium is available in pharmaceutically acceptable purity from Mallinckrodt Specialty Chemicals of St. Louis, Mo. Docusate sodium is adding in appropriated amounts to the suspension for stabilization of the ibuprofen. Preferred ratio amounts of the docustate sodium to the ibuprofen range from about 1:500 to about 1:10 of ibuprofen to docusate sodium, more preferably from about 1:250 to about 1:15, still more preferably from about 1:100 to about 1:20, and most preferably from about 1:50 to about 1:25 of ibuprofen to docusate sodium.

In one preferred embodiment of the present invention, the aqueous oral formulation may include ibuprofen dissolved in water, an effectively stabilizing amount of docusate sodium, a preservative and a viscosity enhancing agent. More preferably, the composition may also contain other conventional excipients such as a sweetener, a flavor and/or flavoring aids.

The present invention may include suitable buffers (also referred to herein as “buffer salts” or “buffering agent”). As used herein, the term “buffers” is intended to mean a compound used to resist a change in pH upon dilution or addition of acid or alkali. Preferred buffer salts include, by way of example and without limitation, potassium dihydrogen orthophosphate, disodium hydrogen orthophosphate, citric acid and disodium hydrogen orthophosphate, potassium phosphate dibasic, sodium phosphate dibasic, potassium metaphosphate, potassium phosphate, monobasic sodium acetate and sodium citrate anhydrous and dehydrate and other such like materials known in the art. Of these, citric acid is preferred. Suitable buffers are generally selected to be chemically unreactive with the other ingredients that may be present in the suspension, with the buffers present in amounts sufficient to provide the desired degree of pH buffering. Preferred pHs of the aqueous formulation of the present invention range from about 6.0 to about 8.0, such as for example from about 6.5 to about 7.5, particularly about 6.8 to about 7.4 and more particularly about 6.8 to about 7.1. Variations and adjustments of the pH of the aqueous formulation are preferably obtained by moderating the addition of the buffer salt(s).

Viscosity enhancing agents may be included in the suspensions of the present invention, as appropriate, as determinable by one skilled in the art, to provide desired flow characteristics to the suspension. The amount of viscosity enhancing agent in the formulation is preferably sufficient to give a solution with a viscosity in the range of 10 to 100 centipoises, with a more preferred viscosity range of from about 20 to 90 centipoises, a still more preferred viscosity range of from about 25 to 75 centipoises, and a most preferred viscosity range of from about 50 to 60 centipoises. Representative viscosity enhancing agents suitable for inclusion in the present invention include, for example without limitation, Xanthan gum, sorbitol, glycerol, sucrose or a cellulose derivative such as carboxymethylcellulose or a salt thereof of a C₁₋₄ alkyl and/or a hydroxy-C₂₋₄ alkyl ether of cellulose, such as methylcellulose, ethylcellulose, hydroxypropylcellulose, hydroxyethylmethylcellulose and hydroxypropylmethylcellulose. Guar Gum is the preferred viscosity enhancing agent.

Representative preservatives suitable for use in the present invention include, for example without limitation, one or more alkyl hydroxybenzoates, such as methyl hydroxybenzoates, ethyl hydroxybenzoates, propyl hydroxybenzoates, butyl hydroxybenzoates and the like. Additional preservatives useful in the present invention include, but are not limited to, sodium benzoate, potassium sorbate, salts of edetate (also know as salts of ethylenediaminetetraacetic acid, or EDTA, such as disodium edetate) and antimicrobial agents including parabens (p-hydroxybenzoic acids esters) such as methylparaben, ethylparaben, propylparaben, butylparaben and the like. The preservatives listed herein are exemplary, with the appropriate preservative and amount of a preservative incorporated into the suspension as determinable by one skilled in the art for compatibility and efficacy of the preservative in a given suspension. Techniques and methods for evaluating preservative efficacy in a given pharmaceutical formulations are readily known in the art. Sodium benzoate is preferred, either alone or together with other preservatives to be added to the present pharmaceutical suspension, although other pharmaceutically acceptable preservatives may be substituted therefor. Preservatives may be included in a given pharmaceutical formulation of the present invention as appropriate, with preferred amounts of up to 1 gram per 100 mL of the suspension. More preferably the preservatives are included in amounts that range of from about 0.10 to about 0.75 grams per 100 mL of the suspension, still more preferably from about 0.15 to about 0.5 grams per 100 mL of the suspension, and most preferably from about 0.20 to about 0.4 grams per 100 mL of the suspension.

Coloring agents also may be incorporated in the suspension of the present invention as determined by one skilled in the art to be appropriate, for chemical compatibility with other ingredients in the suspension and the like. Coloring agents are generally used to provide an appealing color to the suspension. Suitable coloring agents for use in pharmaceutical suspensions are well known in the art. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereof and other such materials known to those skilled in the art.

The pharmaceutical formulation of the present invention preferably contains flavoring agents (herein referred to also as “flavorants”), sweetening agents, and combinations thereof to mask the inherently bitter taste associated with ibuprofen, and thereby improving the palatability of the suspension of the present invention. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the suspension in amounts effective to provide a palatable flavor to the suspension. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with ibuprofen as determinable by one skilled in the art. However, flavoring agents are generally present in the suspension in amounts in the range of from about 0 grams to about 10 grams per 100 mL of the suspension, with preferred amounts of from about 2 grams to about 5 grams per 100 mL. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfame K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. A presently preferred sugar sweetener is sucrose. The amount of sugar sweetener used in the suspension varies with the degree of sweetening desired for the particular formulation as determinable by one skilled in the art, with preferred amounts of sugar sweetener ranging from about 0 grams to about 100 grams sugar sweetener per 100 mL of the suspension, more preferably from about 20 grams to about 95 grams per 100 mL of suspension, still more preferably from 25 grams to about 90 grams sugar sweetener per 100 mL of the suspension, and most preferably from about 30 grams to about 85 grams per 100 mL of suspension. Sugar sweeteners maybe replaced or augmented by water soluble artificial sweeteners, such as the suitable artificial sweeteners previously listed and mixtures thereof. The amount of artificial sweetener used in the suspension may vary to provide an appropriate amount of sweetness to the suspension as determinable by one skilled in the art, generally in amounts similar to those of sugar sweeteners described above. Mixtures of sweetening and/or flavoring agents are preferably used.

The stabilized aqueous pharmaceutical ibuprofen composition of the present invention preferably is administered as a pediatric formulation in appropriate unit dosage form.

A process for producing an aqueous pharmaceutical ibuprofen composition comprises the steps of preparing a solution of ibuprofen and mixing the ibuprofen solution with an effectively stabilizing amount of docusate sodium to form a stabilized ibuprofen suspension. Preparation of the aqueous pharmaceutical composition of the present invention includes ibuprofen stabilized with docusate sodium using ingredients of a purity such that it is suitable for administration to patients. Generally, the pharmaceutical formulation contains at least one conventional pharmaceutical excipient in addition to the docusate sodium and ibuprofen and/or physiologically acceptable salts thereof.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein.

As used herein, the term “treatment”, or a derivative thereof, contemplates partial or complete inhibition of the stated disease state such as, for example, moderate pain, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others. Other examples of such conditions that may be treated include juvenile arthritis, primary dysmenorrhea, signs and symptoms of rheumatoid arthritis and osteoarthritis.

The typical active daily dose of the ibuprofen depends on various factors such as, for example, the individual requirement of each patient, the route of administration, and the disease. An attending physician may adjust the dosage rate based on these and other criteria if he or she so desires. As an example, a suitable oral dosage form may encompass from about 20 to about 200 mg total daily dose, typically administered in one single dose or equally divided doses. A more preferred range is from about 50 mg to about 150 mg total daily dose, and a most preferred range is from about 75 mg to about 100 mg total daily dose. It should be appreciated that daily doses other than those described above may be administered to a subject, as appreciated by an attending physician.

The ibuprofen API may be used as a single active agent, or may be combined with other active agents, vitamins, minerals, dietary supplements, etc. The therapeutic compound(s) contained within the present device can be formulated as its pharmaceutically acceptable salts. As used herein, “pharmaceutically acceptable salts” refers to derivatives of the disclosed compounds wherein the therapeutic compound is modified by reacting it with an acid or base as needed to form an ionically bound pair. Examples of pharmaceutically acceptable salts include conventional non-toxic salts or the quaternary ammonium salts of the parent compound formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. The pharmaceutically acceptable salts of the present invention can be synthesized from the parent therapeutic compound which contains a basic or acidic moiety by conventional chemical methods. Lists of other suitable salts are found in Remington's Pharmaceutical Sciences, 17^{th} ed., Mack Publishing Company, Easton, Pa., 1985, p. 1418, the relevant disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

In addition to the ibuprofen API in the suspension of the present invention, the suspensions may include other pharmaceutical actives in combination with the ibuprofen. Other pharmaceutical actives suitable for use in the present invention include, for example without limitation, acetaminophen, famotidine, pseudoephedrine hydrochloride, chlorpheniramine maleate, astemizole, dextromethorphan hydrobromide, guaifenesin, diphenhydramine hydrochloride, loperamide hydrochloride, simethicone and antacids (such as magnesium oxide, magnesium carbonate, magnesium hydroxide, magnesium trisilicate, magaldrate, aluminum hydroxide and calcium carbonate) and suitable combinations thereof.

The amount of therapeutic compound incorporated in each device of the invention will be at least one or more dosage form and can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of a drug or pharmaceutically active substance which is enough for the required or desired therapeutic response, or in other words, the amount, which is sufficient to elicit an appreciable biological response when, administered to a patient. The appreciable biological response may occur as a result of administration of single or multiple unit doses of an active substance. Depending upon the active substance used and upon the amount of active substance present in a particular device according to the invention, a unit dose may comprise one or more such devices.

### EXAMPLE 1

A stabilized aqueous pharmaceutical ibuprofen composition was formulated by mixing a solution of the ibuprofen together with docusate sodium and other excipients, in purified water. The formulation included 1.33% wt/vol of ibuprofen and 0.05% wt/vol of docusate sodium.

The foregoing summary, description, and examples of the invention are not intended to be limiting, but are only exemplary of the inventive features which are defined in the claims.

What is claimed is:

1. A stabilized pharmaceutical suspension, comprising:
a suspension having:
a pharmaceutically effective amount of ibuprofen; and,
an amount of docusate sodium effective to stabilize the ibuprofen in suspension.

2. The composition of claim 1, wherein the ratio amount of the docustate sodium to the ibuprofen is from about 1:500 to about 1:10 of ibuprofen to docusate sodium.

3. The composition of claim 2, wherein the ratio amount of the docustate sodium to the ibuprofen is from about 1:250 to about 1:15 of ibuprofen to docusate sodium.

4. The composition of claim 3, wherein the ratio amount of the docustate sodium to the ibuprofen is from about 1:100 to about 1:20 of ibuprofen to docusate sodium.

5. The composition of claim 5, wherein the ratio amount of the docustate sodium to the ibuprofen is from about 1:50 to about 1:25 of ibuprofen to docusate sodium.

6. The composition of claim 1, wherein the suspension is substantially free of polyoxyethylene sorbitan monoleate.

7. The composition of claim 1, wherein the suspension is substantially free of pregelatinized starch.

8. The composition of claim 1, further comprising a buffer.

9. The composition of claim 1, further comprising a sweetener.

10. The composition of claim 9, wherein the sweetener is selected from the group consisting of sodium saccharin, sorbitol, sodium cyclamate, sucrose and combinations thereof.

11. A unit dosage of the composition of claim 1, wherein the ibuprofen is present in an amount of up to about 50 grams of ibuprofen per 1000 mL of suspension.

12. The unit dosage of claim 11, wherein the ibuprofen is present in an amount of from about 0.25 grams to about 40 grams of ibuprofen per 1000 mL of suspension.

13. The unit dosage of claim 12, wherein the ibuprofen is present in an amount of from about 0.5 grams to about 25 grams of ibuprofen per 1000 mL of suspension.

14. The unit dosage of claim 13, wherein the ibuprofen is present in an amount of from about 0.75 grams to about 10 grams of ibuprofen per 1000 mL of suspension.

15. The unit dosage of claim 14, wherein the ibuprofen is present in an amount of from about 1 gram to about 5 grams of ibuprofen per 1000 mL of suspension.

16. A process for producing a pharmaceutically acceptable suspension of ibuprofen, comprising the steps of:
preparing an aqueous ibuprofen composition; and,
blending the aqueous ibuprofen composition with an effectively stabilizing amount of docusate sodium to produce a stabilized ibuprofen suspension thereof.

17. The process of claim 16, wherein the ratio amount of the docustate sodium to the ibuprofen is about 1:25 of ibuprofen to docusate sodium.

18. A stabilized aqueous pharmaceutical ibuprofen composition produced by the process of claim 16.

19. A stabilized aqueous pharmaceutical ibuprofen composition produced by the process of claim 17.

20. A pediatric formulation comprising the stabilized pharmaceutical suspension of claim 1.
